(19) United States
(12) Patent Application Publication
(10) Pub. No.: US 2022/0291130 A1
(43) Pub. Date: Sep. 15, 2022

(54) Diagnostic Device Based On Surface-Enhanced Raman Scattering

(71) Applicant: Nanyang Technological University, Singapore (SG)

(72) Inventors: Quan LIU, Xiamen, Fujian (CN); Clement Yuen, Singapore (SG)

(21) Appl. No.: 17/689,834

(22) Filed: Mar. 8, 2022

(30) Foreign Application Priority Data
Mar. 9, 2021 (SG) 10202102374

(51) Int. Cl. G01N 21/65

(52) U.S. Cl. CPC G01N 21/658; G01N 2201/06113

(57) ABSTRACT

Embodiments are directed to diagnostic devices based on surface-enhanced Raman scattering comprising: an inlet module receiving liquid to be analyzed; a reaction module having a first region arranged with a receiving hole and a second region arranged with an output hole, wherein the receiving hole is communicated with the output hole through a flow channel configured with at least one chemical set, the reaction module receives the liquid delivered by the inlet module via the receiving hole, and the liquid to be analyzed flows through the chemical sets placed in the flow channel to obtain nanoparticles-carrying liquid, and the nanoparticles-carrying liquid configured to flow into the second region of the reaction module; and a detection module receiving the nanoparticle-carrying liquid from the output hole of the reaction module.

### CROSS-REFERENCE TO RELATED APPLICATIONS

This application claims priority from the Singapore patent application 10202102374 filed Mar. 9, 2021, the content of which are incorporated herein in the entirety by reference.

### FIELD

The described embodiments relate generally to a diagnostic device based on surface-enhanced Raman scattering.

### BACKGROUND

In 2019, an estimated 229 million cases of human malaria disease and 409,000 related deaths occurred worldwide (W.H.O. 2020). To reduce the morbidity and mortality rates, early malaria diagnosis is critical (Ashley et al. 2018).

The “gold standard” for malaria diagnosis is the microscopic examination of Giemsa-stained blood smears, but this method is time-consuming and requires skilled workers to identify infection especially in cases around the detection limit of 4-20 parasites per μl of blood.

Although a variety of malaria diagnosis techniques have been developed (Ragavan et al. 2018) and attempted to surpass these shortcomings, each of these techniques has its own drawback as well. For example, rapid diagnosis tests (RDTs) have been developed for fast diagnosis in the field that can be used even by any layperson, but these RDTs in general yield lower sensitivity (200 parasites/μl in different stages), a higher chance of false positive or negative results (Reboud et al. 2019), and is unable to perform parasite quantification (Rifaie-Graham et al. 2019) to evaluate the progression of the malaria disease. On the other hand, most of the sensitive methods that allow quantification (e.g., enzyme linked immunosorbent assay, and quantitative real-time polymerase chain reaction) require a laboratory environment for processing (Ashley et al. 2018; Reboud et al. 2019; Rifaie-Graham et al. 2019). Among these techniques, Raman spectroscopy is an optical method that provides vibrational fingerprints for molecular species under examination and shows potential for fast malaria diagnosis without the need of skillful operators, but its insufficient sensitivity makes early diagnosis difficult for low parasitemia level identification (Patel et al. 2019).

Hence, surface enhanced Raman scattering (SERS) has been utilized to improve on the detection sensitivity and augmentation in the Raman signal of hemozoin, a biocrystal that is the unique biomarker of malaria infection (Garrett et al. 2015; Wang et al. 2020; Wood et al. 2011; Yuen and Liu 2013). In the SERS strategy, SERS-active nanoparticles are needed to be brought in close vicinity to hemozoin for Raman signal amplification and most of the SERS studies mixed ready-made nanoparticles with hemozoin obtained from lysed blood prior to SERS measurements to achieve this requirement. Because the approach of complete blood lysis often lyses parasites as well, this step releases and disperses the highly localized aggregated hemozoin biocrystals from each vacuole, into multiple disaggregated biocrystals inside a much larger surrounding volume, which may require further concentrations and extractions (Wang et al. 2020). Otherwise, low SERS signals will be resulted from the increased average distance between nanoparticles and sparsely distributed hemozoin crystals. In contrast, we recently (Chen et al. 2016b) synthesized SERS nanoparticles inside parasites to enhance the Raman signal emitted from hemozoin in malaria infected blood. The advantage of nanoparticle formation inside the parasites is the much improved SERS signal resulting from the close proximity between the SERS nanoparticles and hemozoin inside parasites and/or their vacuoles where hemozoin is highly concentrated. This close proximity is hard to achieve by other ready-made nanoparticles or nanostructures (Garrett et al. 2015; Laing et al. 2017; Perez-Guaita et al. 2018; Wang et al. 2020) because these nanoparticles are not small enough to penetrate through the multiple membrane barriers, e.g., parasite plasma and parasitophorous vacuole membranes, and get close to hemozoin inside.

However, this strategy needs a laboratory environment and bulky equipment (e.g., centrifuge system and ultrasonic bath sonicator) to make the nanoparticles within parasites. These issues make this benchtop method difficult to be used, or scaled up in the field for point-of-care malaria diagnosis, since 95% of global malaria cases (W.H.O. 2020) occur in developing countries, typically in isolated locations with a low-resource setting.

Therefore, we propose a low-cost SERS chip capable of performing on-chip sample preparation and near-analyte nanoparticle synthesis for highly sensitive malaria field diagnosis in this work. This proposed chip allows a user to just add water and a drop of malaria-infected blood to mix with the dried chemicals (constituent reagents for nanoparticles synthesis) deposited earlier in the chip for synthesizing the SERS nanoparticles at close vicinities to hemozoin, prior to SERS measurements. This nanoparticle synthesis methodology is different from another on-chip synthesis approach (Gao et al. 2014), in which their nanoparticles are already formed prior to the mixing with the analyte, i.e. diquat dibromide monohydrate molecules in water. In this manner, our strategy gives a much stronger hemozoin signal and moves one step closer towards the on-site SERS based malaria diagnosis. Moreover, the chip simplifies operation in the process of producing chemical solution precursors thus in turn reducing the risk of contacting corrosive and hazardous chemicals. Another advantage is that our strategy eliminates the issues caused by the limited shelf life of other ready-made SERS substrates, such as flocculation in colloidal substrates, contamination, deterioration, and surface chemistry variations in solid substrates (Perez-Jimenez et al. 2020; Phan and Haes 2019). Most importantly, the chip can be made easily without using any complicated equipment, such as photolithographic techniques, femtosecond-lasers, and electrode position, in contrast to other chips that have been reported to synthesize SERS nanoparticles in situ in the literature.

In addition to reporting the method to fabricate the SERS chip, we optimize the design and configuration of the SERS chip by comparing the performance of various prototypes in terms of the SERS performance for Rhodamine 6G (R6G) and hemozoin measurements. Using the R6G-optimized chip, the SERS enhancement factor and sensitivity of the chip are evaluated with R6G to provide a reference for comparison with other SERS substrates reported in the literature (EI-Zahry et al. 2016; Hidi et al. 2016; Jahn et al. 2017). Lastly, we determine the correlation between representative SERS peak intensities and the corresponding hemozoin concentrations in malaria infected blood acquired by the hemozoin-optimized SERS chip to estimate hemozoin concentrations in unknown samples, which is validated using the partial least squares (PLS) regression, leave-one-out cross validation (LOOCV) analysis.

### SUMMARY

Embodiments described herein are directed to a diagnostic device based on surface-enhanced Raman scattering.

The diagnostic device based on surface-enhanced Raman scattering can include an inlet module receiving liquid to be analyzed; a reaction module having a first region arranged with a receiving hole and a second region arranged with an output hole, wherein the receiving hole is communicated with the output hole through a flow channel configured with at least one chemical set, the reaction module receives the liquid delivered by the inlet module via the receiving hole, and the liquid to be analyzed flows through the chemical sets in the flow channel to obtain nanoparticles-carrying liquid, and the nanoparticles-carrying liquid configured to flow into the second region of the reaction module; and a detection module receiving the nanoparticle-carrying liquid from the output hole of the reaction module, wherein a laser light is configured to be irradiated to the nanoparticles-carrying liquid received by the detection module through the output hole of the reaction module, and is excited to generate the surface-enhanced Raman scattering.

In some cases, the chemical sets comprise a plurality of chemical reagents, each chemical reagent is arranged in the flow channel in the form of dried chemical spots, and each dried chemical spots is arranged at intervals in sequence.

In some cases, the number of the chemical sets is 3 sets or 4 sets, and each chemical sets is arranged at intervals in sequence.

In some cases, the diameter of the receiving hole is larger than the diameter of the output hole.

In some cases, the reaction module comprises a first substrate and a first parafilm stack layer disposed on the first substrate, and a through groove is formed on the first parafilm stack layer to form the flow channel.

In some cases, the inlet module and the detection module are arranged on the same side of the reaction module.

In some cases, the detection module comprises two single-layer parafilms, a fiber glass filter paper and an aluminum foil, the fiber glass filter paper is sandwiched between the two single-layer parafilms and arranged on the aluminum foil, each single-layer parafilm has a hole, and the holes of the two single-layer parafilms are aligned, and nanoparticles, or the nanoparticles and biomarkers of the nanoparticles-carrying liquid delivered through the holes of the two single-layer parafilms from the output hole of the reaction module are deposited on the fiber glass filter paper, and liquid filtered by the fiber glass filter paper flows out from a needle hole of the aluminum foil.

In some cases, the diameter of the holes of the two single-layer parafilms is equal to and larger than the diameter of the output hole.

In some cases, the diameter of the holes of the two single-layer parafilms is equal to and larger than the diameter of the output hole.

In some cases, the aluminum foil is arranged with a needle hole that is not aligned with the holes of the two single-layer parafilms.

In some cases, the size of the fiber glass filter paper is smaller than the sizes of the two single-layer parafilms.

In some cases, the diagnostic device comprises a filtration module, wherein the filtration module is disposed between the inlet module and the first region of the reaction module, to filter the liquid to be analyzed delivered by the inlet module.

In some cases, the filtration module comprises a single-layer parafilm, a second parafilm stack layer and a grade 1 filter paper, the grade 1 filter paper is sandwiched between the single-layer parafilm and the second parafilm stack layer, the second parafilm stack layer is in close contact with the reaction module, the single-layer parafilm has a hole and the second parafilm stack layer has a hole, and the hole of the single-layer parafilm film is aligned with the hole in the second parafilm stack layer.

In some cases, the second parafilm stack layer is formed by stacking four or more than four single-layer parafilms.

In some cases, the first parafilm stacking layer is formed by stacking four or more than four single-layer parafilms.

In some cases, the size of the grade 1 filter paper is smaller than the size of the single-layer parafilm of the filtration module, and smaller than the size of the second parafilm stack layer.

In some cases, the inlet module comprises a cap, a third parafilm stack layer and a second substrate, the cap is arranged on the third parafilm stack layer, the third parafilm stack layer is arranged on the second substrate, the second substrate is in close contact with the single-layer parafilm, and the inlet module has a liquid channel through which the liquid to be analyzed enters into the reaction module from the filtration module.

In some cases, the cap is formed with a through hole, the third parafilm stack layer is formed with a hole, and the second substrate is formed with a needle hole, and the liquid channel of the inlet module is formed with the through hole of the cap, the hole of the third parafilm stack layer and the needle hole of the second substrate.

In some cases, the third parafilm stack layer is formed by stacking eight or more than eight single-layer parafilms.

In some cases, the diagnostic device is used for the diagnosis of malaria infected blood.

In some cases, the nanoparticles are silver nanoparticles.

### BRIEF DESCRIPTION OF THE DRAWINGS

Detailed discussion of implementations directed to one of ordinary skill in the art is set forth in the specification, which makes reference to the appended figures, in which:

FIG. 1 shows a schematic drawing of a diagnostic device/chip based on surface-enhanced Raman scattering and photos of various components.

(a): Partially exploded schematic and the constituent subassembly components of the chip: (I) a reaction module, with 3 sets of dried chemical spots A, B, C, and D; (II) a filtration module; (III) an inlet module; and (IV) a detection module.
(b): Detailed dimension of the reaction module.
(c)-(f): Subassembly photos that corresponding to module (I)-(IV), respectively.
(g): Photo of the entire assembled SERS lab-on-chip.
(h): Photo of our manual syringe pump.

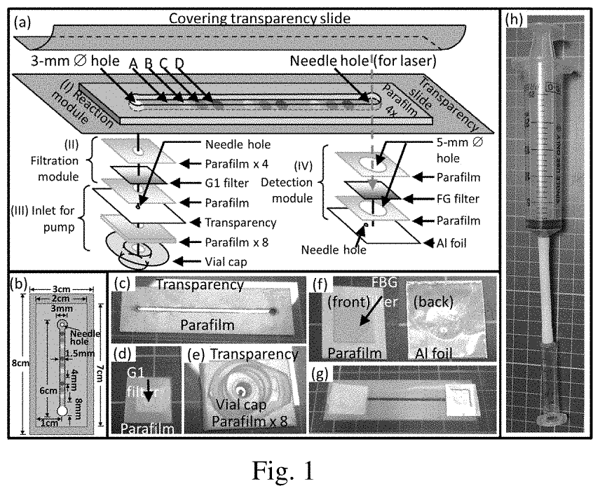

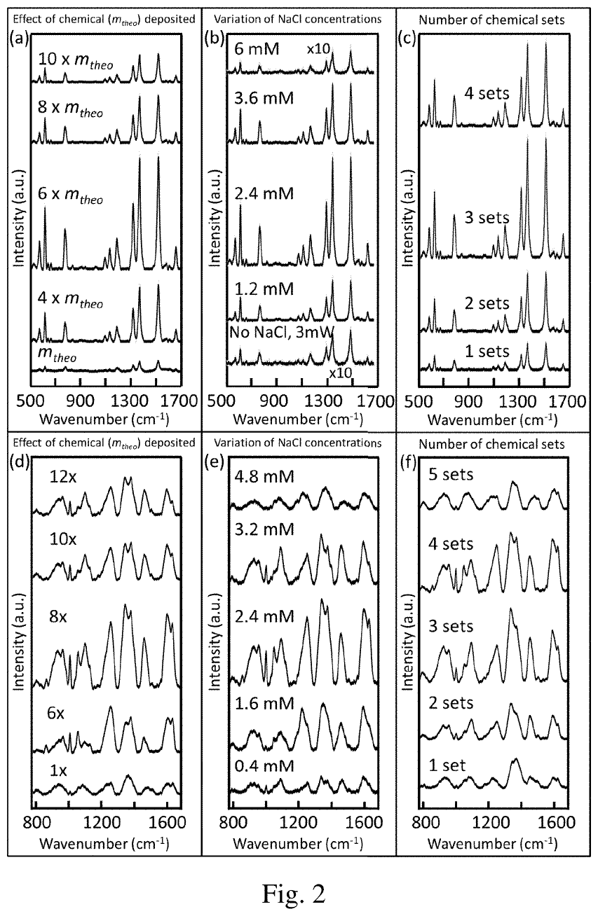

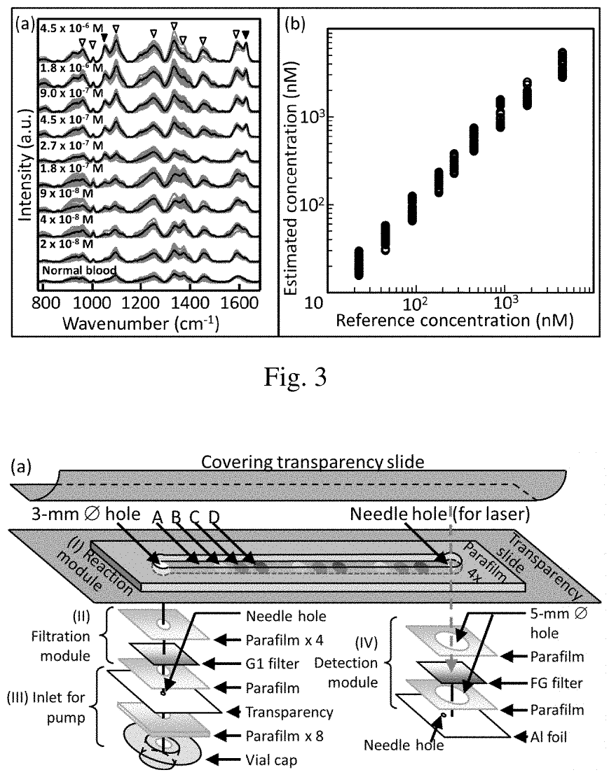

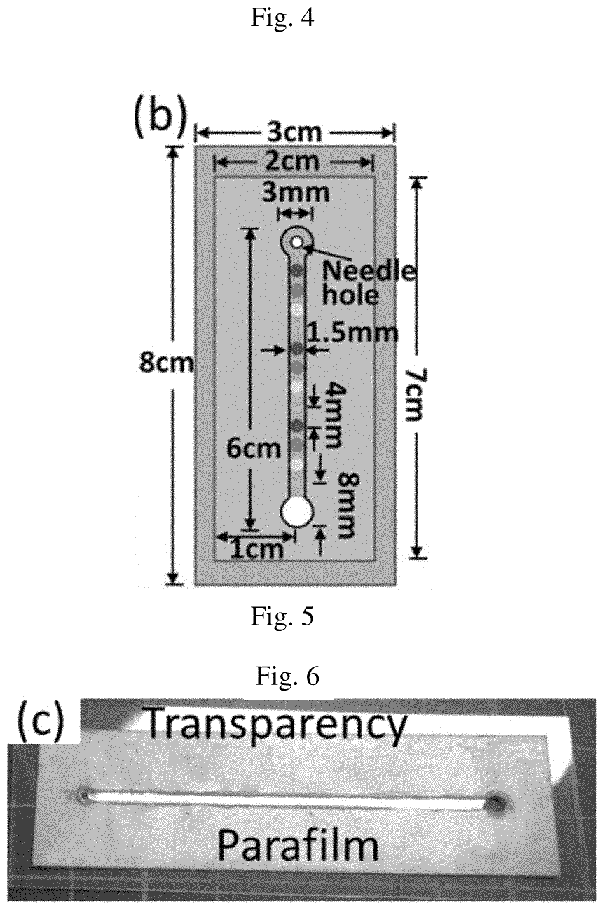

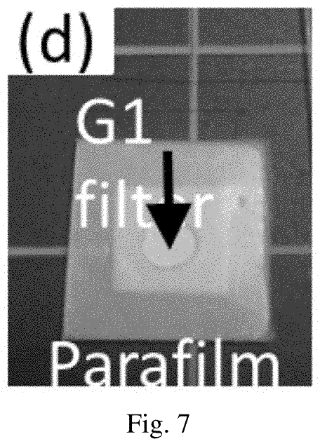

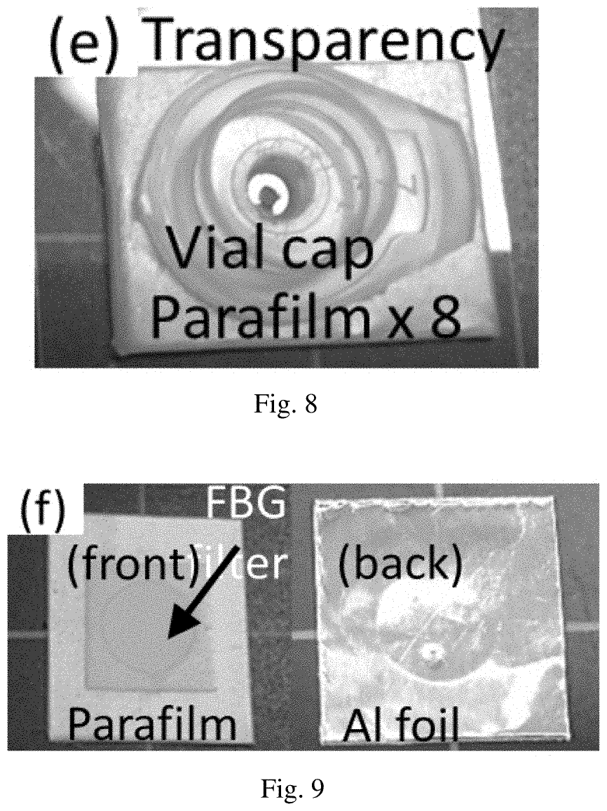

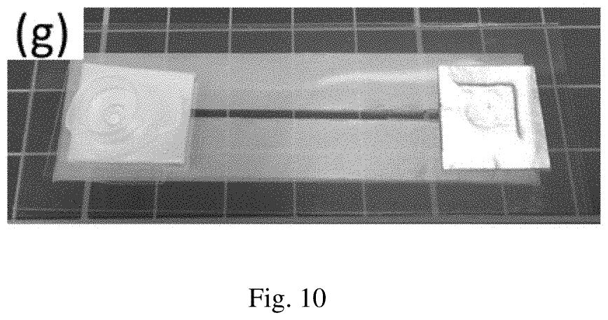

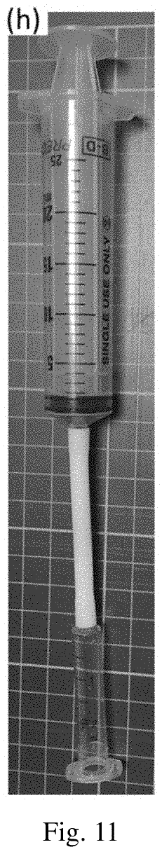

In FIG. 1, A: Silver Nitrate; B: Sodium Hydroxide C: Hydroxylamine Hydrochloride; D: Sodium Chloride; Al: Aluminum; FG filter: Fiber glass filter paper; G1 filter: Grade 1 filter paper. All holes in this chip are 3 mm in diameter (0), unless stated as needle hole (0.81 mm), or hole with diameter of 5 mm. The needle hole on the Al foil in the detection module does not align to the laser path, while the other needle hoe (for laser) on the transparency in the reaction modules in intended for passing the laser beam for SERS measurements.

FIG. 2 shows SERS spectra. SERS spectra of (a)-(c) R6G at concentration of 10⁻⁵M, and (d)-(f) infected blood with parasitemia level of 0.05%, for the optimized chip in comparison to other chips with: (a): 1×, 4×, 6×, 8×, 10× theoretical mass (mₜₕₑₒ), and (d): 1×, 6×, 8×, 10×, 12×mₜₕₑₒ of chemicals deposited; (b): 0 mM, 1.2 mM, 2.4 mM, 3.6 mM, 6 mM, and (e): 0.4 mM, 1.6 mM, 2.4 mM, 3.2 mM, 4.8 mM of NaCl; (c): 1 set, 2 sets, 3 sets, 4 sets, and (f): 1 set, 2 sets, 3 sets, 4 sets, 5 sets of 4 dried chemical spots. The legend “×10” indicates that the intensities of the corresponding spectrum have been multiplied by 10 to facilitate visualization.

FIG. 3 shows comparison between SERS spectra of malaria infected blood and SERS spectra of the normal blood. (a): SERS spectra of malaria infected blood with hemozoin concentrations of 2×10⁻⁸ M, 4×10⁻⁸ M, 9×10⁻⁸ M, 1.8×10⁻⁷ M, 2.7×10⁻⁷ M, 4.5×10⁻⁷ M, 9.0×10⁻⁷M, 1.8×10⁻⁸ M, and 4.5×10⁻⁸ M, in comparison to that of the normal blood. Each average spectrum (black) was averaged from the 25 spectra (grey) acquired from 5 different samples with 5 random locations each. (b): Correlation between estimated hemozoin concentrations and reference hemozoin concentrations, in which the former values were evaluated by the PLS-LOO technique from SERS spectra acquired using the hemozoin-optimized chip.

FIGS. 4-11 are enlarged views of part (a)-part (h) of FIG. 1, respectively.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a”, “an”, and “the” include plural referents unless the context clearly dictates otherwise. Thus, for example, reference to “a component surface” includes reference to one or more of such surfaces.

Throughout the specification, when an element is referred to as being “connected to” another element, it may be directly or indirectly connected to the other element and the “indirectly connected to” includes connected to the other element via a wireless communication network.

In addition, the terms used in the specification are merely used to describe particular embodiments of the disclosure, and are not intended to limit the disclosure. In addition, it is to be understood that the terms, such as “comprise”, “include”, “have”, or the like, are intended to indicate the existence of the features, numbers, operations, components, parts, or combinations thereof disclosed in the specification, and are not intended to preclude the possibility that one or more other features, numbers, operations, components, parts, or combinations thereof may exist or may be added.

It will be understood that, although the terms “first”, “second”, etc., may be used herein to describe various elements, these elements should not be limited by these terms. The above terms are used only to distinguish one component from another. For example, a first component discussed below could be termed a second component, and similarly, the second component may be termed the first component without departing from the teachings of this disclosure.

Hereinafter, embodiments of the disclosure will be described with reference to the accompanying drawings.

Materials and Methods

Chemicals and Materials

Silver nitrate (AgNO₃), rhodamine 6G, (R6G), sodium chloride (NaCl), Percoll, sorbitol, and sodium hydroxide (NaOH) pellets were ordered from Merck, Germany. RPMI 1640 medium, and AlbuMAX® were purchased from ThermoFisher, USA. Hydroxylamine hydrochloride (HONH2.HCl) was purchased from MP Biomedicals, USA. Parafilm PM996 M roll, Grade 1 filter paper, and glass microfiber filters (Grade GFfB) were purchased from Whatman, United Kingdom. Transparency film (PP2500) was bought from 3M, United States. Silhouette Cameo 4 cutting machine was bought from Silhouette, USA.

Ethics Statement

The whole blood was donated by healthy non-malarial immune adult volunteers at the National University Hospital, Singapore. Informed written consents were obtained from all donors in accordance with protocols approved by Institutional Review Board of Nanyang Technological University, Singapore (IRB-2018-02-031).

Plasmodium falciparum Parasite Culture

P. falciparum parasite strain 3D7 were cultured in fresh RBCs at 4% hematocrit in RPMI 1640 supplemented with 5% albumax with 3% O₂, 5% CO₂, and balance with N2 gas and incubated at 3TC as previously described (Trager, W, Jensen, J. B., 1976. Human malaria parasites in continuous culture. Science 193(4254), 673-675). Growth media was replaced every day with fresh complete RPMI. Smears were made on microscope slide to check the parasitemia. Late schizont stage parasites were purified using 70% Percoll centrifugation as described (Kutner, S., et al, 1985). For tighter synchronization, the purified schizonts stage parasites were allowed to reinvade in fresh RBCs. After 5 to 6 h of growth, the parasites were treated with 5% sorbitol to remove all late-stage parasites (Aley, S. B., et al, 1986) such that 96-99% of the parasites were in the ring stage. The blood sample was stored in 4 degree Celsius while all measurements were completed in about 30 days from the end of parasite culturing.

Embodiments of the diagnostic device based on surface-enhanced Raman scattering are discussed below with reference to FIGS. 1 and 4.

The diagnostic device may include an inlet module, a reaction module and a detection module. The inlet module may be used to receive liquid to be analyzed. The reaction module may have a first region arranged with a receiving hole and a second region arranged with an output hole. The output hole may be a needle hole for receiving a laser. The receiving hole may be communicated with the output hole through a flow channel. The flow channel may be configured with at least one chemical set. The reaction module may receive the liquid delivered by the inlet module via the receiving hole. The liquid to be analyzed flows through the chemical sets arranged in the flow channel to obtain nanoparticles-carrying liquid. The nanoparticles-carrying liquid can flow into the second region of the reaction module.

The detection module is used to receive the nanoparticle-carrying liquid from the output hole of the reaction module. A laser light is irradiated to the nanoparticles-carrying liquid received by the detection module through the output hole of the reaction module, and is excited to generate the surface-enhanced Raman scattering.

The nanoparticles described herein are preferably Ag nanoparticles, or Au nanoparticles, etc. The skilled person in the art should understand that other types of nanoparticles can also be used in the disclosure.

The Ag nanoparticles are discussed below as example of nanoparticles to describe the embodiments of the disclosure.

The liquid to be analyzed described herein may be human blood or animal blood.

The human blood or animal blood may be delivered to the reaction module of the diagnostic device through the inlet module. The reaction module can be arranged with the flow channel. The chemical sets located in the flow channel can be dissolved by the human blood or animal blood. The dissolved chemical sets carry out a chemical reaction to generate Ag nanoparticles. The Ag nanoparticles can exist in the form of silver colloid. Thus, the human blood or animal blood to be analyzed is mixed with the Ag nanoparticles.

The Ag nanoparticles-carrying liquid is irradiated by a laser light, and form surface-enhanced Raman scattering. The diagnosis on whether the liquid includes biomarkers indicating a specific disease such as malaria can be conducted, by obtaining the Raman spectroscopy and making spectroscopy analysis. By this way, on-site malaria diagnosis will be achieved.

Preferably, the chemical sets of the diagnostic device of the disclosure include a plurality of chemical reagents. Each chemical reagent is arranged in the flow channel in the form of dried chemical spots, and the dried chemical spots are arranged at intervals in sequence.

The chemical sets may include Silver Nitrate, Sodium Hydroxide, Hydroxylamine Hydrochloride, and Sodium Chloride. The 4 chemical reagents may be arranged at intervals in sequence in the flow channel. The interval between two adjacent chemical reagent may be equal.

Preferably, the number of the chemical sets may be 3 sets or 4 sets, and the chemical sets may be arranged at intervals in sequence.

In our experiments, the Raman scattering is best when the chemical sets is 3 sets or 4 sets. However, the skilled person should understand that the number of the chemical sets and the included chemical reagents may be adjusted.

The diameter of the receiving hole may be larger than the diameter of the output hole. Based on the diameter arrangement, the liquid to be analyzed can dissolve completely the dried chemical spots.

With reference to FIGS. 1 and 5, the reaction module may include a first substrate and a first parafilm stack layer disposed on the first substrate. A through groove is formed on the first parafilm stack layer to form the flow channel as described herein.

the “substrate” described in the present disclosure may be a sheet-like substrate of various materials, such as a sheet-like substrate made of PVC (Polyvinyl chloride), and the “substrate” described in the present disclosure is preferably a transparent substrate. Under the inspiration of the technical solutions of the present disclosure, those skilled in the art can select or adjust the material, type, etc. of the “substrate”, which all fall within the protection scope of the present disclosure.

According to an embodiment of the present disclosure, the receiving hole is provided on a first substrate and communicated with a first end of the through groove, and the output hole is provided on the first substrate and communicated with a second end of the through groove.

According to a preferred embodiment of the present disclosure, referring to FIGS. 1 and 5, both the first end of the through groove and the second end of the through groove are in a circular shape or an oval shape.

The reaction module of the diagnostic device of the present disclosure further includes a third substrate, and the first parafilm stack layer is sandwiched between the first substrate and the third substrate.

As shown in FIG. 1, the inlet module and the detection module of the diagnostic device of the present disclosure are arranged on the same side of the reaction module.

Referring to FIGS. 1 and 9, the detection module of the diagnostic device preferably includes two single-layer parafilms, a fiber glass filter paper and an aluminum foil. The fiber glass filter paper is sandwiched between the two single-layer parafilms and arranged on the aluminum foil. Each single-layer parafilm has a hole, and the holes of the two single-layer parafilms are aligned. Nanoparticles, or the nanoparticles and biomarkers of the nanoparticles-carrying liquid delivered through the holes of the two single-layer parafilms from the output hole of the reaction module are deposited on the fiber glass filter paper, and liquid filtered by the fiber glass filter paper flows out from a needle hole of the aluminum foil.

Referring to FIG. 1, the diameters of the holes of the two single-layer parafilms are equal, and are larger than the diameter of the output hole.

Referring to FIGS. 1 and 9, a needle hole is preferably provided on the aluminum foil of the detection module, and the pinhole on the aluminum foil is not aligned with the circular holes on the two single-layer parafilms of the detection module.

Preferably, the diameter of the fiber glass filter paper of the detection module may be smaller than the diameter of the single-layer parafilm of the detection module.

By setting the diameter of the fiber glass filter paper of the detection module of the diagnostic device to be smaller than the diameter of the single-layer parafilm, the fiber glass filter paper can be completely placed between the two single-layer parafilms.

The diagnostic device may further include a filtration module. The filtration module may be arranged between the inlet module and the first region of the reaction module, so as to filter the liquid to be analyzed delivered by the inlet module.

According to one preferred embodiment of the present disclosure, the filtration module of the diagnostic device may include a single-layer parafilm, a second parafilm stack layer and a grade 1 filter paper (G1 filter). The grade 1 filter paper may be sandwiched between the single-layer parafilm and the second parafilm stack layer. The second parafilm stack layer may be in close contact with the reaction module. The single-layer parafilm has a hole and the second parafilm stack layer has a hole, and the hole of the single-layer parafilm film is aligned with the hole in the second parafilm stack layer.

The second parafilm stack layer may be formed by stacking four or more than four single-layer parafilms.

The first parafilm stack layer of the reaction module of the diagnostic device may be formed by stacking four or more than four single-layer parafilms.

Under the inspiration of the technical solutions of the present disclosure, those skilled in the art can adjust or select the number of single-layer parafilms of the second parafilm stack layer, and adjust or select the number of single-layer parafilms of the first parafilm stack layer.

Referring to FIG. 1, the diameter of the G1 filter of the filtration module may be smaller than the diameter of the single-layer parafilm of the filtration module and smaller than the diameter of the second parafilm stack layer of the filtration module.

Referring to FIGS. 1 and 4, the inlet module of the diagnostic device may include a cap, a third parafilm stack layer and a second substrate. The cap may be arranged on the third parafilm stack layer. The third parafilm stack layer may be arranged on the second substrate. The second substrate may be in close contact with the single-layer parafilm of the filtration module. The inlet module may have a liquid channel through which the liquid to be analyzed enters into the reaction module from the filtration module.

Referring to FIGS. 1, 4 and 8, the cap of the inlet module may be formed with a through hole. The third parafilm stack layer may be formed with a hole. The second substrate is formed with a needle hole. The liquid channel of the inlet module is formed with the through hole of the cap, the hole of the third parafilm stack layer and the needle hole of the second substrate.

The third parafilm stack layer is formed by stacking eight or more than eight single-layer parafilms. Under the inspiration of the technical solutions of the present disclosure, those skilled in the art can adjust or select the number of single-layer parafilms of the third parafilm stack layer of the inlet module, which all fall within the protection scope of the present disclosure.

The diagnostic device as described in various embodiments may preferably further include a housing (not shown). The assembled reaction module, filtration module, inlet module and detection module may be placed in the housing.

According to one embodiment of the disclosure, the diagnostic device/chip may be prepared by the following steps.

Firstly, the prepared materials (filter paper, vial cap, Aluminum (Al) foil, parafilms and transparency) are patterned in the desired shapes and dimensions (as shown in FIGS. 1(a) and 1(b)) by using the Silhouette Cameo paper cutting machine. The diameters of holes diameter may be 3 mm, unless otherwise stated.

Subsequently, the subassembly components are made as follows.

REACTION MODULE: Four pieces of parafilms are stacked together to form a first parafilm stack layer. Each piece of parafilm is patterned with two holes (for example 3 mm) connected by a channel to form the reaction module. The first parafilm stack layer is positioned onto a first substrate (transparency slide). The first substrate has a first hole and a second hole. The diameter of the first hole is preferably 3 mm. The second hole is preferably a 21-G needle hole. The first hole is aligned with one hole of the two holes of the first parafilm stack layer, and the second hole is aligned with the other hole of the two holes of the first parafilm stack layer, as shown in FIGS. 1(b) and (c).

FILTRATION MODULE: Four pieces of parafilms are stacked together to form a second parafilm stack layer. A grade 1 filter paper is sandwiched between the second parafilm stack layer and the single-layer parafilm to form the filtration module. The second parafilm stack layer is formed with a hole, and the single-layer parafilm is also formed with a hole. The hole of the second parafilm stack layer is aligned with the hole of the single-layer parafilm.

INLET MODULE (Inlet for pump): A hole was punched into the vial cap, and preferably 3 mm in diameter. The vial cap may be formed by cutting off from a vial. The vial cap is disposed on a third parafilm stack layer (see FIG. 1(e)). The third parafilm stack layer is disposed on a second substrate (such as transparency slide). The third parafilm stack layer may be formed by stacking eight pieces of parafilms.

DETECTION MODULE: A piece of fiber glass filter paper (see FIG. 1(f), preferably with a size of 7 mm×7 mm) is sandwiched between two pieces of parafilms (each with a 5 mm round hole that is aligned to each other), and placed onto a sheet of Al foil with a needle hole (misaligned from the central axis formed from holes in the two aligned parafilms). The misaligned needle hole is for releasing of pressure built up by analyte at the detection module during pumping and minimizing the draining of nanoparticles and parasites.

All the aforesaid subassembly components are aligned and assembled (FIG. 1(a)). A sacrificial sheet of Al foil is used to cover the channel (temporarily in place of the covering transparency slide) and this assembled device/chip is heated to 120° C. on a hot plate for four minutes. Note that this assembly should be positioned such that the sacrificial Al foil is in contact with the surface of the hot plate during the heating process. Chemicals are not placed in the channel in this step due to the high temperature.

The sacrificial Al foil is removed and chemicals are dropped in the channel. AgNO₃ (20.5 μg) is dropped at a location of 8 mm away from the edge of the channel, followed by NaOH (14.4 μg), HONH₂.HCl (12.5 μg and NaCl (2.8 μg) with 4 mm distance apart from each other (see FIGS. 1(a) and 1(b), positions indicated by A, B, C, D).

These chemicals are air dried in a dark environment. To seal up, a third substrate (transparency slide) is covered on top (see FIG. 1(a)) and heated on a hot plate at 60° C. for four minutes to melt the parafilm to adhere onto the third substrate. The first parafilm stack layer is sealed between the first substrate and the third substrate. This temperature is sufficiently low to prevent any unwanted chemical reactions in the dried patches before usage (see FIG. 1(g)), since a much higher temperature is needed for those reactions, e.g. thermal decomposition of AgNO₃ takes place at 160° C.

Operation of the Device/Chip for SERS Measurement Preparation

In each test, 20 μl test analyte is input into the inlet (inlet module) of the chip for pumping. The vial cap is plugged into the barrel flange of our self-made syringe pump (see FIG. 1(h)). The analyte is forced to advance to the spot with dried AgNO₃ (Point A, first spot) and allowed to re-dissolve the chemical for two minutes before moving onto the next chemical spot (Point B, second spot). The advancement of the analyte to the next dried spot is visually monitored by the operator through the transparency and can be assisted with markings on the syringe. This timing procedure was also applied to processing the rest of the dried chemical spots (Third spot C till the twelfth spot, in FIG. 1(a)), and the analyte is pushed into the detection module in the end. Prior to a SERS measurement, the covering transparency slide and the parafilm are peeled off. A laser light (dotted arrow shown in FIG. 1(a)) is focused onto the fiber glass filter paper through the needle hole (for laser) on the transparency slide for the SERS measurement.

R6G concentration of 10⁻⁵ M and malaria infected blood with parasitemia level of 0.05% are used to find the optimal chemical configuration for the R6G and hemozoin SERS measurement. In R6G SERS measurements, 20 μl aqueous R6G is used as the test analyte. In malaria related SERS measurements, 10 μl deionized water is mixed with 10 μl normal or malaria-infected blood with various parasitemia levels (ring stage) of 0%, 0.0025%, 0.005%, 0.01%, 0.02%, 0.03%, 0.05%, 0.1%, 0.2%, and 0.5%, which corresponds to hemozoin concentrations of 0 M, 2×10⁻⁸M, 4×10⁻⁸ M, 9×10⁻⁸M, 1.8×10⁻⁷M, 2.7×10⁻⁷M, 4.5×10⁻⁷M, 9.0×10⁻⁷M, 1.8×10⁻⁶ M, and 4.5×10⁻⁶ M, respectively, as the test analyte sequentially. The equivalent hemozoin concentrations were evaluated by assuming that human body contains 5×10⁹ red blood cells (RBCs) per milliliter, hemozoin concentrations of 0.22 μg/RBC in the ring stage and a molecular weight of 1229 g/mol for hemozoin.

Raman Instrumentation and Data Processing

We evaluated the Raman characteristics of our devices by employing a compact micro-Raman system (innoRam-ySSS, B&W TEK, US) coupled to a video microscope (BAC151A, B&W TEK, US) in a backscattered configuration. In our SERS measurements, a 785 nm laser (at 5 mW for blood measurements and 1 mW for R6G measurements, unless stated otherwise) was focused onto the sample through an objective lens (60×, N.A. 0.85) attached to the video microscope. Upon excitation, Raman signals emitted from samples were collected by the same objective and diffracted by a grating with a spectral resolution of 4 cm⁻¹ for detection. Each spectrum was acquired with an exposure time of 20 s and accumulated for 4 times. To obtain the final spectra, each raw spectrum underwent five-point moving average to remove noise prior to the removal of fluorescence background. The displayed spectrum in the result section was averaged from these final spectra acquired from 5 different samples with 5 random locations each (unless otherwise stated). Moreover, we modeled the SERS spectrum of malaria-infected blood from 1535 cm⁻¹ to 1645 cm⁻¹ as the superposition of contributions from two vibrational features of v, CₐCₘ (1586 cm⁻¹ in blood) and v_{C=C} (1624 cm⁻¹ in hemozoin). To calculate the Raman signal contribution from hemozoin alone (v_{C=C} at 1624 cm⁻¹), we fit the segment as the summation of two Lorentzian functions and calculated the area under the fitted Lorentzian curve from 1622 cm⁻¹ to 1626 cm⁻¹.

Results

SERS Performance Optimization of the Device/Chip with Different Amounts of Chemicals

FIG. 2 shows the SERS performance for two different sets of chips with different amount of chemicals optimized for the SERS measurement of (FIG. 2(a)-FIG. 2(c)) R6G, and (FIG. 2(d)-FIG. 2(f)) infected blood.

FIG. 2(a)-FIG. 2(d) evaluate the variations in SERS spectra of R6G and hemozoin, respectively, for different amounts of AgNO₃, NaOH, and HONH₂.HCl deposited onto the dried chemical spots. The theoretical mass [mₜₕₑₒ) of chemicals were 3.4 μg of AgNO₃ (1 mM), 2.4 of NaOH (3 mM), and 2.1 of HONH₂.HCl (1.5 mM) for 20 μl-volume analyte. Different amounts of chemicals with mass in multiples of mₜₕₑₒ were deposited in chips, correspondingly, for R6G and hemozoin SERS measurements. The SERS results gave an optimal SERS improvement for chip deposited with 6×mₜₕₑₒ in R6G analyte and 8×mₜₕₑₒ in hemozoin analyte. FIG. 2(b)-FIG. 2(e) study the different NaCl concentrations in chips for R6G and hemozoin tests, respectively. With an NaCl concentration of 2.4 mM, the SERS intensities for both R6G and hemozoin (FIG. 2(b) and FIG. 2(e)) showed the best results. FIG. 2(e) and FIG. 2(f) illustrate the SERS spectra of R6G and hemozoin analytes, respectively, that flowed through different sets of the four dried-chemical spots were also examined. The results showed that 3 sets (FIG. 2(c), and 4 sets (FIG. 2(f) of the dried chemicals rendered the highest R6G and hemozoin SERS intensities, respectively. Therefore, we observed that the R6G-optimized chip with 6-time theoretical mass of chemical deposited, 2.4 mM of NaCl, and 3 sets of dried chemicals offered the best R6G SERS signals. We also noted that the hemozoin-optimized chip with 8-time theoretical mass of chemical deposited, 2.4 mM of NaCl, and 4 sets of dried chemicals produced the highest hemozoin SERS signals.

Malaria Diagnosis by Quantifying Hemozoin Concentration in Malaria Infected Blood

FIG. 3(a) shows the SERS spectra of uninfected blood and malaria infected (ring stage) blood when the parasitemia level was varied from 0.0025% to 0.5%, using the hemozoin-optimized chip. Distinct SERS Raman peaks (“∇” in FIG. 3(a)) at 951 cm⁻¹ (v₃CH₃), 1003 cm⁻¹ (v₄₇), 1087 cm⁻¹ (v_{c-c}), 1247 cm⁻¹ (Amide III), 1345 cm⁻¹ (C_{2vinyi}H), 1375 cm⁻¹ (v₄), 1448 cm⁻¹ (δ, CH₂/CH₃), and 1584 cm⁻¹ (v, CₐCₘ) were present, which were similar to other vibrational features reported (Atkins et al. 2017; Chen et al. 2016a; Chen et al. 2016b) in the literature to be found in both malaria infected blood and normal blood. Moreover, prominent peaks (“▾” in FIG. 3(a)) at 1053 cm⁻¹ (unavailable assignment) and 1624 cm⁻¹ (v_{c=c}) were also observed in the malaria infected blood, comparable to SERS results (Chen et al. 2016b) that were obtained using a laboratory-based SERS method. FIG. 3(b) plots the estimated concentrations against the reference concentrations of hemozoin using the PLS-LOOCV technique with a RMSEP of 0.3 μm based on the SERS peak at 1624 cm⁻¹. We also found that the lowest detectable parasitemia level was 0.0025% in the ring stage, or hemozoin concentrations of 20 nM, based on a series of t-test (p<0.05 by comparing the peak at 1624 cm⁻¹ with that from the normal blood sample).

### DISCUSSION

We have improved the SERS performance of our chip by optimizing the amounts of chemicals (FIG. 2) in the following. First, the incomplete solvation of chemicals in the chip was compensated by increasing the masses of chemicals with the same ratio, the highest SERS signal was noted in 6×mₜₕₑₒ (FIG. 2(a)) and 8×mₜₕₑₒ (FIG. 2(d)) of chemicals deposited. In fact, we expected this ratio to vary for the different types of chemicals and to be different for the same type chemical in the different set of dried spots (e.g., spot 1, 5, and 9 for AgNO₃), but for this preliminary study we assumed all to be the same. Secondly, NaCl was introduced into the chip (FIGS. 2(b) and 2(e)) to induced Ag nanoparticles aggregations and the formations of nano-gaps for further SERS augmentation, similar to others reported in the literature (Han et al. 2011; Min et al. 2018) for other SERS structures and applications. These geometries allowed more effective SERS activities in contrast to the relatively sparsely distributed Ag nanoparticles. Thirdly, the analyte flowed through three chemical sets (FIG. 2(c)) and four chemical sets (FIG. 2(f)) gave the optimal improvement, which was probably due to the enlargement of Ag-nanoparticle size as the analyte flowed through more sets of dried chemicals, similar to the strategy (Yuen and Liu 2013) used by us to grow other types of enlarged Ag structures. The larger diameter Ag nanoparticles exhibited a higher value of extinction cross-section in the NIR wavelength (Yu et al. 2017; Yuen and Liu 2013), resulting in a higher SERS intensity. After the optimal set number, the reduction of SERS intensities was likely due to the further size increment in Ag nanoparticles with reduced the nanoparticles density per hemozoin. Hence, we have augmented the SERS performance of with two different chip configurations based on the two different test molecules: R6G (FIGS. 2(a)-(c)) in water and hemozoin (FIGS. 2(d)-(f)) in blood. These results demonstrated the feasibility of the on-site instant synthesis of SERS active nanoparticles on a chip for the sensitive field measurements of chemical and biological analyte molecules.

Furthermore, we characterized the SERS performance of R6G molecules, using the optimized chip. The R6G-optimized chip effectively augmented the SERS signal of R6G, in contrast to the spontaneous Raman signal. The analytical enhancement factor (AEF) of our chip can be found to be comparable to other Ag nanoparticles fabricated in a laboratory environment (in the range from 4×10³ to 7×10⁵) (Cañamares et al. 2008; Ju et al. 2017) using a similar approach (Leopold, N., et al. 2003), and to other types of SERS chips (Zhao et al. 2016) with nanoparticles formed in-situ. Moreover, the SERS intensities correlated well to the R6G concentration with a root-mean-square error of prediction (RMSEP) value of 49 when applying the PLS-LOOCV technique (equation A.2), which was comparable to other SERS chips (Yaghobian et al. 2011) reported. Thus, we realized the sensitive chemical SERS measurements by this facile near-analyte synthesis of Ag nanoparticles on a chip, eliminating the issue of shelf life existing in other types (Perez-Jimenez et al. 2020) of SERS substrates, due to the instant synthesis characteristic. This methodology was also shown capable for SERS testing of biological molecules, hemozoin (FIG. 3).

We also investigated the detection of hemozoin concentration in the malaria infected blood by utilizing this SERS chip (FIG. 3). FIG. 3(a) illustrates SERS spectra of the malaria infected and uninfected blood acquired by our hemozoin-optimized chip. We noted that the vibrational features originated from the hemozoin biocrystal (e.g., v_{c=c}) were more prominent in our SERS measurement than other studies (Chen et al. 2016a). It is worth noting that blood is lysed on the chip using only a small amount of water in an attempt to keep a considerable amount of the malaria biomarker, hemozoin, confined in parasites and/or their vacuoles to achieve locally high concentrations. The stronger SERS signal from hemozoin is likely due to the diffusion of aqueous AgNO3 and other chemicals through the multiple membrane barriers, which enables the formation of nanoparticles nearer to the aggregated hemozoin biocrystals located within the vacuoles for extra SERS augmentation. Conversely, other groups reported that the SERS spectra characteristics of the membrane or other blood components were dominated (with lesser contribution from the hemozoin biomarker) in case of no lysing (Chen et al. 2016a). Otherwise, a separate step of lysing all membranes was needed to expose hemozoin crystals (Garrett et al. 2015), which could lead to the release of aggregated hemozoin biocrystals within the vacuole and dispersed into a much larger volume after lysing, thus reducing its localized concentration and in turn the SERS signal. Therefore, our strategy is promising for potential on-site malaria diagnosis by analyzing the SERS spectra, without the requirement of lab environment and complicated procedures (e.g., step of lysing blood). FIG. 3(b) evaluated the area under the fitted Lorentzian curve from 1622 cm⁻¹ to 1626 cm⁻¹ as described earlier, to study the unique vibrational mode (v_{c=c}) contributed by the hemozoin biocrystal for quantifying the parasitemia level to assess the progression of the malaria disease. The RMSEP value of 0.3 μm (equation A.2) in our result (FIG. 3(b)) was better than other types of SERS chip (RMSEP of 4 μM) reported (Morelli et al. 2018) for bacteria detection in the literature. AEF was not discussed since hemozoin was difficult to detect inside the parasite without Ag nanoparticles even at a high parasitemia level. Moreover, if cells and parasites were to be totally lysed to expose hemozoin biocrystals, the situation would be different from the highly possible existence of unlysed parasites and/or their vacuoles in our experiment. The detection limit of 0.0025% is equivalent to about 125 parasites/μl (with the assumption that normal blood in human body contains 5×10⁹ RBCs/ml) in the ring stage, or a detection limit of 42 parasites/μl for detection of parasites in the schizont stage. This estimation is based on the fact that the hemozoin concentration of parasites in the schizont stage is approximately three times that in the ring stage (Serebrennikova et al. 2010). Yet, this detection limit was different from that reported in our previous work (Chen et al. 2016b) (0.00005%) performed in a laboratory setting. The discrepancy in detection limit could be attributed to several potential factors. First, the dried chemicals might be incompletely dissolved into the blood sample due to insufficient mixing. In addition, the Raman system for reading the SERS chip was a more compact and cost effective Raman system innoRam-785S (B&W TEK, US) in contrast to the Renishaw system (inVia, Renishaw, UK) in our previous work (Chen et al. 2016b); however, at the cost of sensitivity reduction. Another important factor affecting the sensitivity was the excitation wavelength of 785 nm in this study, which was different from 633 nm used in our previous work. For silver nanoparticles of the same size, the extinction coefficient under 633 nm excitation was expected about 3 times higher than that under the 785 nm excitation, based on the extinction cross-section calculation of Mie light scattering by a single Ag nanoparticles (Abajo 2019; Yu et al. 2017), thereby the detection limit can be theoretically improved by 3 folds (about 14 parasites/μl in the schizont stage).

In contrast to other SERS methodologies, the near-analyte and instant synthesis of SERS nanoparticles inside our chip improves the shelf-life and minimize required sample preparation procedures, e.g. no need of a centrifuge to concentrate hemozoin, which is similar to RDTs. However, our chip can achieve a higher sensitivity compared to common RDT techniques, and has the advantage of quantifying hemozoin to indicate the severity and progression of the malaria disease in a patient. Additionally, our chip needs neither the stringent laboratory environment nor expensive equipment, e.g. cleanroom conditions and facilities, for chip operation and fabrication, which lowers the running and manufacturing cost of the chip compared to most laboratory-based techniques. Therefore, the further development of this method and a low-cost optical spectrometer could yield an effective technique based on SERS for malaria diagnosis with high sensitivity in the field.

In conclusion, we develop a SERS fluidic chip for the sensitive measurements of hemozoin towards malaria field diagnosis with a detection limit of 0.0025% parasitemia level, i.e. 125 parasites/μl, in the ring stage, which could be improved further by another three folds by simply switching the laser wavelength. This chip can be operated in the field without a laboratory environment, and with minimized handling of hazardous chemical precursors. It is worth noting that the chip can be easily fabricated and mass-produced at a low cost. More importantly, SERS active nanoparticles are instantaneously synthesized in the presence of blood with hemozoin (biomarker) inside the chip, which achieves the formation of near-analyte nanoparticles for stronger SERS signals and longer shelf life. Therefore, our strategy advances SERS-based malaria diagnosis closer to low-cost point-of-care field testing on a large scale.

### REFERENCES

Abajo, F. J. G. d., 2019. Mie light scattering by a single sphere: Extinction cross section. http://widgets.nanophotonics.es/mie/index.html.
Aley, S. B., Sherwood, J. A., Marsh, K., Eidelman, O., Howard, R. J., 1986. Identification of isolate-specific proteins on sorbitol-enriched Plasmodium falciparum infected erythrocytes from Gambian patients. Parasitology 92(3), 511-525.
Ashley, E. A., Phyo, A. P., Woodrow, C. J., 2018. Malaria. Lancet 391(10130), 1608-1621.
Atkins, C. G., Buckley, K., Blades, M. W., Turner, R. F. B., 2017. Raman spectroscopy of blood and blood components. Appl. Spectrosc. 71(5), 767-793.
Cañamares, M. V., Garcia-Ramos, J. V., Sanchez-Cortes, S., Castillejo, M., Oujja, M., 2008. Comparative SERS effectiveness of silver nanoparticles prepared by different methods: A study of the enhancement factor and the interfacial properties. J. Colloid Interface Sci. 326(1), 103-109.
Chen, F., Flaherty, B. RV Cohen, C. EV Peterson, D. S V Zhao, Y., 2016a. Direct detection of malaria infected red blood cells by surface enhanced Raman spectroscopy. Nanomedicine 12(6), 1445-1451. Chen, K., Yuen, C., Aniweh, Y., Preiser, P., Liu, Q., 2016b. Towards ultrasensitive malaria diagnosis using surface enhanced Raman spectroscopy. Sci. Rep. 6, 20177.
EI-Zahry, M. R., Refaat, I. H., Mohamed, H. A., Lendl, B., 2016. Sequential SERS determination of aspirin and vitamin C using in situ laser-induced photochemical silver substrate synthesis in a moving flow cell. Anal. Bioanal. Chem. 408, 4733-4741.
Gao, R., Choi, N., Chang, S. I., Lee, E. K., Choo, J., 2014. Real-time analysis of diaquat dibromide monohydrate in water with a SERS-based integrated microdroplet sensor. Nanoscale 6(15), 8781-′8786.
Garrett, N. L., Sekine, R., Dixon, M. W., Tilley, L., Bambery, K. R., Wood, B. R., 2015. Bio-sensing with butterfly wings: naturally occurring nano-structures for SERS-based malaria parasite detection. Phys. Chem. Chem. Phys. 17(33), 21164-21168.
Han, X., Goebl, J., Lu, Yin, Y., 2011. Role of salt in the spontaneous assembly of charged Gold nanoparticles in ethanol. Langmuir27(9)/5282-5289.
Hidi, J. I., Jahn, M., Weber, K., Bocklitz, T., Pletz, M. W., Cialla-May, D., Popp, J., 2016. Lab-on-a-chip-surface enhanced raman scattering combined with the standard addition method: Toward the quantification of nitroxoline in spiked human urine samples. Anal. Chem. 88(18), 9173-9180.
Jahn, I. JV Zukovskaja, O., Zheng, X. SV Weber, Kv Bocklitz, T. W., Cialla-May, D., Popp, J., 2017. Surface-enhanced Raman spectroscopy and microfluidic platforms: challenges, solutions and potential applications. Analyst 142(7), 1022-1047.
Ju, J., Liu, W., Perlaki, C. M., Chen, K., Feng, C., Liu, Q., 2017. Sustained and cost effective Silver substrate for surface enhanced Raman spectroscopy based biosensing. Sci. Rep. 7(1), 6017.
Kutner, S., Breuer, W. V., Ginsburg, H., Aley, S. B., Cabantchik, Z. I., 1985. Characterization of permeation pathways in the plasma membrane of human erythrocytes infected with early stages of Plasmodium falciparum: association with parasite development. J. Cell Physiol. 125(3), 521-527.
Laing, S., Jamieson, L. E., Faulds, K., Graham, D., 2017. Surface-enhanced Raman spectroscopy for in vivo biosensing. Nat. Rev. Chem. 1, 0060.
Leopold, N., Lendl, B., 2003. A new method for fast preparation of highly surface-enhanced Raman scattering (SERS) active silver colloids at room temperature by reduction of silver nitrate with hydroxylamine hydrochloride. J. Phys. Chem. B 107(24), 5723-5727.
Min, K., Jeon, W. J., Kim, Y., Choi, J. Y., Yu, H. K., 2018. Spontaneous nano-gap formation in Ag film using NaCl sacrificial layer for Raman enhancement. Nanotechnology 29(10), 105502.
Morelli, L., Serioli, L., Centorbi, F. A., Jendresen, C. B., Matteucci, M., Ilchenko, O., Demarchi, D., Nielsen, A. T., Zor, K., Boisen, A., 2018. Injection molded lab-on-a-disc platform for screening of genetically modified E. coli using liquid-liquid extraction and surface enhanced Raman scattering. Lab. Chip. 18(6), 869-877.
O'Haver, T., 2016. A Pragmatic Introduction to Signal Processing. Lulu Enterprises Incorporated.
Patel, S. KV Rajora, Nv Kumar, S., Sahu, Av Kochar, S. KV Krishna, C. M., Srivastava, S., 2019. Rapid discrimination of Malaria- and Dengue-infected patients sera Using Raman spectroscopy. Anal. Chem. 91(11), 7054-7062.
Perez-Guaita, D., Marzec, K. M., Hudson, A., Evans, C., Chernenko, T., Matthaus, C., Miljkovic, M., Diem, M., P. Heraud, Richards, J. S., Andrew, D., Anderson, D. A., Doerig, C., Garcia-Bustos, J., McNaughton, D., Wood, B. R., 2018. Parasites under the spotlight: Applications of vibrational spectroscopyto malaria research. Chem. Rev. 118(11), 5330-5358.
Perez-Jimenez, A. I., Lyu, D., Lu, 2., Liu, G., Ren, B., 2020. Surface-enhanced Raman spectroscopy: benefits, trade-offs and future developments. Chem. Sci. 11(18), 4563-4577.
Phan, H. T., Haes, A. J., 2019. What does Nanoparticle stability mean? J. Phys. Chem. C 123(27), 16495-17062.
Ragavan, K. V., Kumar, S., Swaraj, S., Neethirajan, S., 2018. Advances in biosensors and optical assays for diagnosis and detection of malaria. Biosens. Bioelectron. 105, 188-210.
Reboud, J., Xu, G., Garrett, A. f Adriko, M., Yang, Z., Tukahebwa, E. M., Rowell, C., Cooper, J. M., 2019. Paper-based microfluidics for DNA diagnostics of malaria in low resource underserved rural communities. Proc. Natl. Acad. Sci. U.S.A 116(11), 4834-4842.
Rifaie-Graham, O., Pollard, J., Raccio, S., Balog, S., Rusch, S., Beck, M.A.H.-C.P.Y.M.H.P., Bruns, N. f 2019. Hemozoin-catalyzed precipitation polymerization as an assay for malaria diagnosis. Nat. Commun. 10(1), 1369.
Serebrennikova, Y. M., Patel, J., Garcia-Rubio, L. H., 2010. Interpretation of ultraviolet-visible spectra of malaria parasite Plasmodium falciparum. Appl. Opt. 49(2), 180-188.
Stern, K. H., 1972. High Temperature Properties and Decomposition of Inorganic Salts Part 3, Nitrates and Nitrites. J. Phys. Chem. Ref. Data 1(3), 747-772.
Trager, W., Jensen, J. B., 1976. Human malaria parasites in continuous culture. Science 193(4254), 673-675.
W.H.O., 2017. World malaria report 2018.
W.H.O., 2020. World malaria report 2020.
Wang, W., Dong, R., Gu, D., He, J., Yi, P., Kong, S., Ho, H., Loo, J. F., Wang, W., Wang, Q., 2020. Antibody-free rapid diagnosis of malaria in whole blood with surface-enhanced Raman spectroscopy using nanostructured gold substrate. Adv. Med. Sci. 65(1), 86-92.
Wood, B. R., Bailo, E., Khiavi, M. A., Tilley, L., Deed, S., Gaudig, T. D., McNaughton, D., Deckert, V., 2011. Tip-enhanced Raman scattering (TERS) from hemozoin crystals within a sectioned erythrocyte. Nano Lett. 11(5), 1868-1873.
Yaghobian, F., Weimann, T., Guttler, By Stosch, R., 2011. On-chip approach for traceable quantification of biomarkers based on isotope-dilution surface-enhanced Raman scattering (IDSERS). Lab. Chip. 11(17), 2955-2960.
Yu, R., Liz-Marzan, L. M., Abajo, F. J. G. d., 2017. Universal analytical modeling of plasmonic nanoparticles. Chem. Soc. Rev. 46(22), 6710-6724.
Yuen, C., Liu, Q., 2012. Magnetic field enriched surface enhanced resonance Raman spectroscopy for early malaria diagnosis. J. Biomed. Opt. 17(1), 017005.
Yuen, C., Liu, Q., 2013. Optimization of Fe3O4@Ag nanoshells in magnetic field-enriched surface-enhanced resonance Raman scattering for malaria diagnosis. Analyst 138(21), 6494-6500.
Zhang, Z. M., Chen, S., Liang, Y. Z., Liu, Z. X., Zhang, Q. M., Ding, L. X., Ye, F., Zhou, H., 2009. An intelligent background—correction algorithm for highly fluorescent samples in Raman spectroscopy. J. Raman Spectrosc. 41(6), 659-669.
Zhao, H. Z., Xu, Y., Wang, C. Y., Wang, R., Xiang, S. T., 2016. Design and fabrication of a microfluidic SERS chip with integrated Ag film@nanoAu. RSC Adv. 6(17), 14105-14111.

In the description of the present disclosure, the description with reference to the terms “an embodiment”, “some embodiments”, “example”, “specific example”, or “some examples” and the like means that specific features, structures, materials or characteristics described in connection with the embodiments or examples are included in at least one embodiment or example of the present disclosure. In the present specification, the schematic expressions of the above terms are not necessarily directed to the same embodiments or examples. Furthermore, the specific features, structures, materials, or characteristics described may be combined in a suitable manner in any one or more embodiments or examples. In addition, various embodiments or examples described in the specification, as well as features of various embodiments or examples, may be combined by those skilled in the art without causing any contradiction.

While the embodiments of the disclosure have been shown and described above, it can be understood that the foregoing embodiments are illustrative and are not to be construed as limiting the present application. Variations, amendments, substitutions and modifications may be made by those ordinarily skilled in the art to the foregoing embodiments within the scope of the present application.

What is claimed is:

1. A diagnostic device based on surface-enhanced Raman scattering, comprising:
an inlet module receiving liquid to be analyzed;
a reaction module having a first region arranged with a receiving hole and a second region arranged with an output hole, wherein the receiving hole is communicated with the output hole through a flow channel configured with at least one chemical set, the reaction module receives the liquid delivered by the inlet module via the receiving hole, and the liquid to be analyzed flows through the chemical sets placed in the flow channel to obtain nanoparticles-carrying liquid, and the nanoparticles-carrying liquid configured to flow into the second region of the reaction module; and
a detection module receiving the nanoparticle-carrying liquid from the output hole of the reaction module,
wherein a laser light is configured to be irradiated to the nanoparticles-carrying liquid received by the detection module through the output hole of the reaction module, and is excited to generate the surface-enhanced Raman scattering.

2. The diagnostic device of claim 1, wherein the chemical sets comprise a plurality of chemical reagents, each chemical reagent is arranged in the flow channel in the form of dried chemical spots, and each dried chemical spots is arranged at intervals in sequence.

3. The diagnostic device of claim 2, wherein the number of the chemical sets is 3 sets or 4 sets, and each chemical set is arranged at intervals in sequence.

4. The diagnostic device of claim 1, wherein the diameter of the receiving hole is larger than the diameter of the output hole.

5. The diagnostic device of claim 1, wherein the reaction module comprises a first substrate and a first parafilm stack layer disposed on the first substrate, and a through groove is formed on the first parafilm stack layer to form the flow channel.

6. The diagnostic device of claim 1, wherein the inlet module and the detection module are arranged on the same side of the reaction module.

7. The diagnostic device of claim 1, wherein the detection module comprises two single-layer parafilms, a fiber glass filter paper and an aluminum foil,
the fiber glass filter paper is sandwiched between the two single-layer parafilms and arranged on the aluminum foil,
each single-layer parafilm has a hole, and the holes of the two single-layer parafilms are aligned, and
nanoparticles, or the nanoparticles and biomarkers of the nanoparticles-carrying liquid delivered through the holes of the two single-layer parafilms from the output hole of the reaction module are deposited on the fiber glass filter paper, and liquid filtered by the fiber glass filter paper flows out from a needle hole of the aluminum foil.

8. The diagnostic device of claim 7, wherein the diameters of the holes of the two single-layer parafilms are equal, and are larger than the diameter of the output hole.

9. The diagnostic device of claim 7, wherein the aluminum foil is arranged with a needle hole that is not aligned with the holes of the two single-layer parafilms.

10. The diagnostic device of claim 7, wherein the size of the fiber glass filter paper is smaller than the sizes of the two single-layer parafilms.

11. The diagnostic device of claim 1, further comprising a filtration module, wherein the filtration module is disposed between the inlet module and the first region of the reaction module, to filter the liquid to be analyzed delivered by the inlet module.

12. The diagnostic device of claim 11, wherein the filtration module comprises a single-layer parafilm, a second parafilm stack layer and a grade 1 filter paper,
the grade 1 filter paper is sandwiched between the single-layer parafilm and the second parafilm stack layer,
the second parafilm stack layer is in close contact with the reaction module,
the single-layer parafilm has a hole and the second parafilm stack layer has a hole, and the hole of the single-layer parafilm film is aligned with the hole in the second parafilm stack layer.

13. The diagnostic device of claim 12, wherein the second parafilm stack layer is formed by stacking four or more than four single-layer parafilms.

14. The diagnostic device of claim 5, wherein the first parafilm stacking layer is formed by stacking four or more than four single-layer parafilms.

15. The diagnostic device of claim 12, wherein the size of the grade 1 filter paper is smaller than the size of the single-layer parafilm of the filtration module, and smaller than the size of the second parafilm stack layer.

16. The diagnostic device of claim 11, wherein the inlet module comprises a cap, a third parafilm stack layer and a second substrate,
the cap is arranged on the third parafilm stack layer,
the third parafilm stack layer is arranged on the second substrate,
the second substrate is in close contact with the single-layer parafilm, and
the inlet module has a liquid channel through which the liquid to be analyzed enters into the reaction module from the filtration module.

17. The diagnostic device of claim 16, wherein the cap is formed with a through hole, the third parafilm stack layer is formed with a hole, and the second substrate is formed with a needle hole, and
the liquid channel of the inlet module is formed with the through hole of the cap, the hole of the third parafilm stack layer and the needle hole of the second substrate.

18. The diagnostic device of claim 17, wherein the third parafilm stack layer is formed by stacking eight or more than eight single-layer parafilms.

19. The diagnostic device of claim 1, wherein the diagnostic device is used for the diagnosis of malaria infected blood.

20. The diagnostic device of claim 1, wherein the nanoparticles are Ag nanoparticles.
